(19) United States
(12) Patent Application Publication
(10) Pub. No.: US 2002/0026181 A1
(43) Pub. Date: Feb. 28, 2002

(54) Method of evaluating surgical laser

(76) Inventor: Francis E. O'Donnell JR., St. Louis, MO (US)

Correspondence Address: PAUL M. DENK, 763 South New Ballas Rd., St. Louis, MO, 63141, US

(21) Appl. No.: 09/931,500

(22) Filed: Aug. 16, 2001

Related U.S. Application Data
(63) Continuation-in-part of application No. 08/647,603, filed on May 13, 1996, now Pat. No. 5,713,893.
Application No. 08/647,603 is a continuation-in-part of application No. 08/410,494, filed on Mar. 24, 1995, now abandoned.
Application No. 08/410,494 is a continuation-in-part of application No. 08/514,806, filed on Oct. 31, 1995, now abandoned.
Application No. 08/514,806 is a continuation-in-part of application No. 08/269,139, filed on Jun. 30, 1994, now Pat. No. 5,460,627.
Application No. 08/269,139 is a continuation-in-part of application No. 08/055,578, filed on May 3, 1993, now abandoned.

(51) Int. Cl. A61B 18/20

(52) U.S. Cl. 606/10; 606/2; 606/5

(57) ABSTRACT

Evaluation of a laser used in ophthalmologic surgery. A test substrate is formed from a homogenous material of controlled water content that ablates at a rate approximately that of human corneal tissue. A first non-contact surface topographical analysis of the test substrate is performed prior to laser ablation. Laser ablation is performed on the test substrate. A second non-contact surface topographical analysis of the test substrate is performed. The second surface topographical analysis is compared with the first to evaluate the surgical laser. The analysis is used to calibrate the laser. The data also is used to enhance the predictive value of a self-educating neural network. The non-contact surface topographical analysis is performed by rasterstereography, videokeratography, laser holography, scanning laser ophthalmoscope, optical coherence tomography, and scanning white light interferometry.

### CROSS-REFERENCE TO RELATED APPLICATION

This application is a continuation-in-part of co-pending application Ser. No. 08/647,603, filed May 13, 1996, which is a continuation-in-part of application Ser. No. 08/410,494, filed Mar. 24, 1995, which is a continuation-in-part of application Ser. No. 08/514,806, filed Oct. 31, 1995, which is a continuation-in-part of application Ser. No. 08/269,139, filed Jun. 30, 1994, now U.S. Pat. No. 5,460,627, issuing Oct. 24, 1995, which is a continuation-in-part of application Ser. No. 08/055,578, filed May 3, 1993.

### BACKGROUND OF THE INVENTION

This invention relates generally to the evaluation of a laser used in ophthalmologic surgery and, more specifically, to a methods of testing the effect of ablations to evaluate and adjust the laser.

Ultraviolet (UV) laser evaluation and calibration typically has been done on a flat substrate of polymethylmethacrylate (PMMA) which is then read on a lensometer to confirm the laser calibration. In my U.S. Pat. No. 5,460,627, I disclose the use of a rastostereographic or placido-disc corneal topographic analysis of a test substrate to enhance the calibration process. Such technologies enhance the calibration process by providing additional information beyond the means of a lensometer. Further, my co-pending application Ser. No. 08/514,806, filed Aug. 14, 1995, further provides for improvements in the above technologies.

Rastostereographs or rastophotogrammetry has been used in opththalmological surgery to measure the surface contour of the optic nerve head and to measure corneal surface curvatures. In the technique of rastophotogrammetry a series of parallel lines or a grid is projected on the surface to be measured. Computerized digital analysis of a video image is performed to detect elevations or depressions of the surface being measured. Rastostereographic imaging is combined with image processing computer software to produce the topography of the cornea, for example.

Recently is has been determined that placido-disc videokeratoscopy can be used to determine surface contour, particularly to visualize and determine the surface contour of a contact lens or artificial cornea. The placido-disc video keratoscope is a type of computerized videokeratography now available to ophthalmologic surgeons. The instrument allows the surgeon to measure and modify corneal curvature. The basic videokeratograph instrument includes a light source projected onto the cornea. The modification of the light by the cornea is captured by a video camera and the information is analyzed by computer software. The data is displayed in a variety of formats including photographs and on a screen.

As stated above, the rastophotogram and placido-disc technologies have been used to analyze a test substrate, rather than the cornea, to evaluate the ablating effect of a surgical laser. And while these technologies enhance the calibration process, they are still limited by the relative inefficiency of UV lasers to ablate test substrates, as opposed to the human cornea. For example, a minus four (4) diopter ablation in cornea can result in as little as one (1) diopter ablation in PMMA. Such a small correction makes it even more difficult to detect calibration error. Specifically, a 2% error would only be 0.24 microns of central ablation depth for a 6 mm ablation zone diameter in PMMA. It would be advantageous, therefore, to utilize other non-contact technologies for determining corneal or substrate topography in order to more accurately evaluate the ablating power and laser effect of a surgical laser.

### SUMMARY OF THE INVENTION

It is among the principal objects of the present invention to provide a method of evaluation of a surgical laser.

Another object of the invention is to provide non-contact techniques for determine the surface profile of the cornea.

Another object of the invention is to provide non-contact techniques for determine the surface profile of the cornea to evaluate the laser effect of a surgical laser.

Still another object of the invention to provide a method of doing a non-contact profilemetric analysis of a substrate so as to not distort the substrate.

Yet another object of the invention is to provide a method of using laser calibration data to improve the predictive value of self-educating neural net-work.

Still another object of the invention is to provide such a substrate that can be analyzed pre-ablation and post-ablation so as to allow evaluation of the ablating laser.

Yet another object of the invention is to provide such a substrate that can be analyzed with a lensometer, rastophotogrammetry, placido-disc videokeratoscopy , laser holography, optical coherence tomography, scanning laser ophthalmoscope, or scanning white light interferometry to give an accurate evaluation of the ablative power of the surgical laser.

Another object of the invention to provide a method of confirming the desired lens effect of a laser beam on a target substrate.

In accordance with the invention, generally stated, as target substrate is provided that is efficiently ablated has characteristics nearly those of human cornea in response to laser energy. The topography of the novel target substrate can be analyzed by rastostereogrammetry, rastophotogrammetry, placido-disc videokeratoscopy, lensometry, laser holography, optical coherence tomography, scanning laser ophthalmoscope, or scanning white light interferometry or other technologies. A test ablation is performed on a substrate. The topographical analysis is repeated. The surgeon can use a comparison of the two topographical analyses to evaluate the laser's effect on human cornea prior to a surgical procedure.

The calibration data are used to improve the predictive value of a self-educating neural network.

### BRIEF DESCRIPTION OF THE DRAWINGS

FIG. 1 is a diagram illustrating the use of rastophotogrammetry to determine unwanted lens effect (i.e. hot or cold spots) of a laser beam;

FIG. 2 is a diagram illustrating the use of rastophotogrammetry to confirm the desired lens effect of a laser beam;

FIG. 3 is a diagram illustrating the use of rastophotogrammetry in an ophthalmologic surgical procedure;

FIG. 4A is a diagram illustrating the use of rastophotogrammetry to evaluate a laser based on the effect on a contact lens of a known refractive power;

FIG. 4B is a diagram illustrating the use of placido-disc videokeratoscopy to evaluate a laser based on the effect on an artificial cornea of a known refractive power;

FIG. 5 is a diagram illustrating the use of rastophotogrammetry to change the refractive power of an intraocular implant;

FIG. 6 is a diagram illustrating use of rastostereophotogrammetry to evaluate the effect of a laser on an artificial cornea of a know refractive power; and

FIG. 7 is a diagram illustrating the use of rastostereophotogrammetry to evaluate the effect of a laser on an anhydrous pliant substrate of the present invention.

### DESCRIPTION OF THE PREFERRED EMBODIMENT

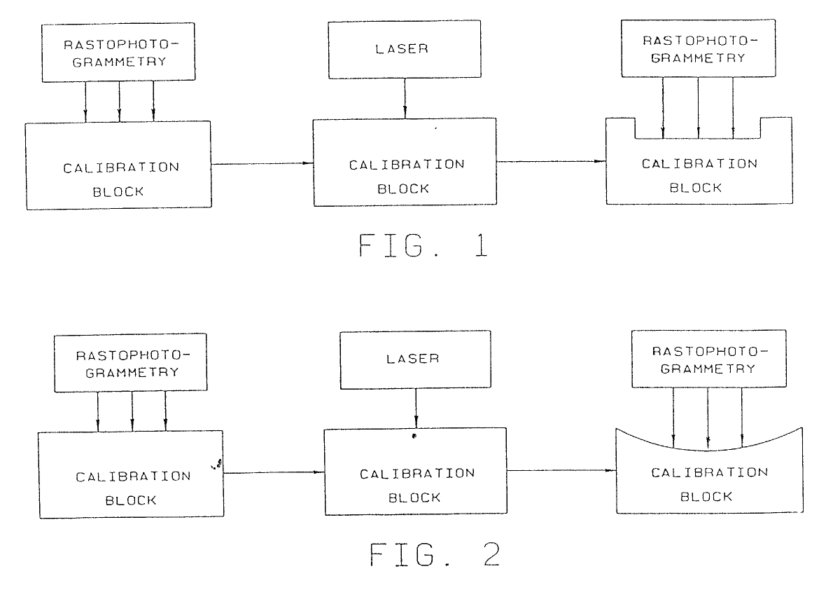

As illustrated in FIG. 1, rastophotogrammetry is used to determine the amount of unwanted lens effect delivered by an excimer or other ultraviolet or infrared laser beam. It should be noted, that although rastophotogrammetry is illustrated throughout the specification as a method of evaluating the topography of a test substrate or cornea, it will be appreciated that the illustration can relate to other techniques for evaluating the surface topography, as will be discussed below.

Rastophotogrammetry devices used in these procedures are the type marketed by PAR Technology Corp., Hartford, N.Y. 13413. A calibration block, typically made of polymethylmethacrylate (PMMA) is employed, in one embodiment. However, improved target substrates, which are the subject of the instant invention, may be used, as will be explained in detail below. The rastophotogrammetry is used before calibration to evaluate the ablative effects of the laser beam. A rastophotogram is made of the calibration block, the laser energy is applied to the calibration block and, a second rastophotogram is made to see if the laser effect is a uniform ablation with no depressions (hot spots) or elevations (cold spots). A shown in FIG. 1, the rastophotogram confirms a uniform and accurate ablation of the calibration block.

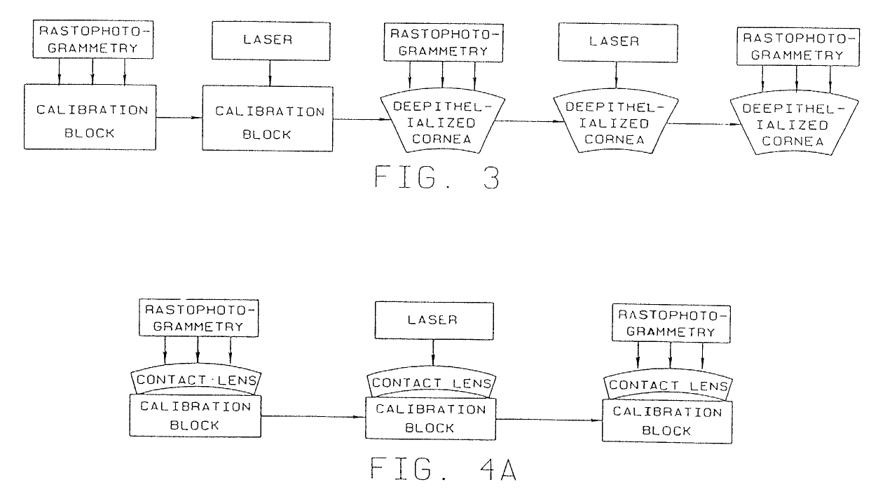

Confirmation of a desired lens effect is illustrated in FIG. 2. A rastophotograrn is made of a target substrate, i.e. a calibration block. The laser beam is applied to the calibration block, and a second rastophotogram is performed to confirm the desired lens effect in the calibration block. For example, the desired lens effect is 4.00 dioptric, the amount of calibration block material that is removed at each point along the radius of the calibration block can be computed and the actual amount removed compared quantitatively and qualitatively to the effect desired. Moreover, the astigmatic and multizone (asphericity) correction desired can be calibrated and measured quantitatively.

FIG. 3 illustrates the use of rastophotogrammetry in corneal surgery performed to alter the refractive power of the human cornea. As illustrated, a rastophotogram is made of a calibration block. As previously stated, the laser is applied to the calibration block to determine when or not there is unwanted lens effect. In this manner and through these steps it can be evaluated whether the laser is properly calibrated before the laser is used on the human eye. Next, a rastophotogram is performed on a deepithelialized human cornea. The laser then is applied to the deepithelialized cornea and ablation performed. Finally, rastophotogrammetry is performed to determine if the proper shape (refractive power) of the deepithelialized cornea has been achieved. The steps may be repeated to validate repeated ablations.

As illustrated in FIG. 3, under-correction can be avoided by continuing the treatment session until the desired amount of correction is achieved. Furthermore, in order to enhance the quality of the photo image at this point, a surface dye can be applied to the deepithelialized cornea. In addition, the rastophotogrammetry grid can be projected using various wave lengths and filters for optimal visualization of the projected grid.

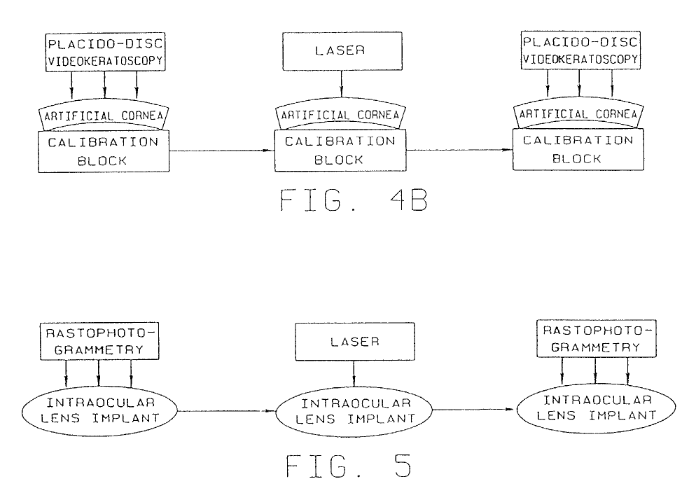

FIG. 4A illustrates the use of rastophotogrammetry in the evaluation of the surgical laser using a target substrate formed as a contact lens of a known dioptric power. A contact lens of a known dioptric power from PMMA is fastened to a holding block. The contact lens can be white to facilitate imaging with the rastophotogrammetric system. Alternatively, it can be impregnated with a fluorescein dye. Next, rastophotogrammetry is performed on the contact lens front surface to get an accurate baseline reading of the topography of the front surface of the lens. Next, the laser is used on the front surface of the lens. Finally, a second rastophotogram is performed to determine that the effect of the laser on the lens results in the desired change in the known dioptric power of the lens so that the laser can be calibrated properly based upon the evaluation of the effect of the laser on a lens with known dioptric power. The steps in the procedure may be repeated to validate the calibration.

FIG. 4B illustrates the use of placido-disc videokeratoscopy in the evaluation of the ablative effects of a surgical laser using an artificial cornea of a known dioptric power formed from PMMA or other appropriate material, as will be described below relative to the instant invention. The artificial cornea is fastened to a holding block, as previously described. The artificial cornea can be black plastic to facilitate imaging with a videokeratoscope. The artificial cornea should be colored all the way through so that ablation does not penetrate only a colored layer.

Next, a placido-disc videokeratoscope procedure is performed on the artificial cornea front surface to get an accurate baseline reading of the topography of the front surface. The placido-disc technology, similar to the rastophotogrammetry, is merely illustrative of a non-contact surface topography assessment method. A first videokeratograph of the topography is made. Next, a laser is used on the front surface of the artificial cornea. A nonviscous, fine hydrophobic lubricant, such as a thin oil, can be placed on the ablated surface of the artificial cornea to enhance visualization since the ablated surface can lose its reflectivity. A second placido-disc videoscope procedure is performed and a second videokeratograph is made. The first and second videokeratographs are compared to evaluate the ablative effective of the laser.

It will be appreciated by those skilled in the art that placido-disc videokeratoscopic procedures work poorly on deepithelialized cornea right after surgery. However, the procedure works well for imaging the artificial cornea or other target substrates before and after an ablation, as first described. Therefore, the placido-disc can be used in connection with rastophotogrammetry in the surgical setting. The effect of laser on an artificial cornea can be determined by the use of placido-disc videokeratoscopy. The laser is used to perform corneal ablation. Subsequently, rastophotogrammetry is performed on the deepithelialized cornea to determine corneal topography. Corneal ablation is performed and a repeat rastophotogram is made and compared to the first to validate the laser effect.

FIG. 5 illustrates the use of the rastophotogram to determine the effective laser adjustment of refractive power of an intraocular lens implant. As illustrated, the rastophotogrammetry procedure is performed on the intraocular implant so as to determine a baseline. Next, the laser beam is applied to the intraocular lens implant to alter the curvature of the implant and thereby alter the refractive power. The second rastophotogram of the intraocular implant is then made to provide a feedback mechanism to ascertain if the desired refractive change has been made. The procedure may be repeated until the desired change in refractive power is achieved.

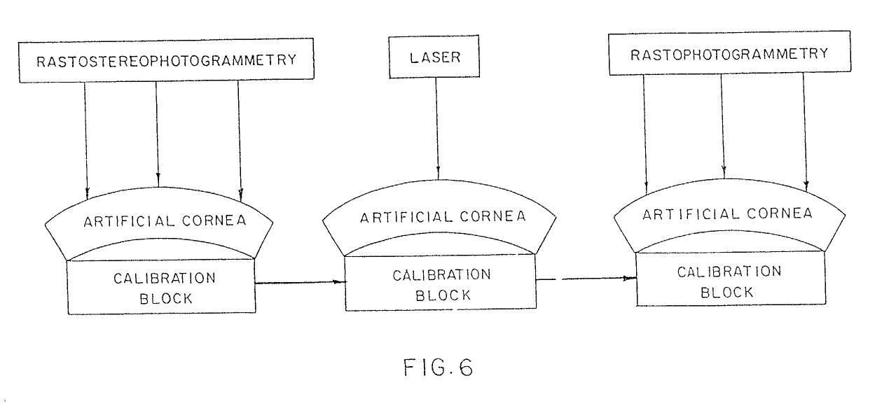

FIG. 6 illustrates the use of rastostereophotogrammetry, in addition to rastophotogrammetry, in the evaluation of a surgical laser using an artificial cornea of a known dioptric power. An artificial cornea of a known dioptric power formed from PMMA or other materials, as will be described below, is fastened to a holding block, as previously described. The artificial cornea can be white or fluorescein-impregnated, all the way through, to facilitate the imaging with the laser.

Following this, the rastostereophotogrammetry procedure is performed on the artificial cornea front surface to get an accurate baseline reading of the topography of the front surface of the artificial cornea. A first rastostereophotogram of the topography is made. Next, a laser is used on the present curvature of the artificial cornea. A rastophotogrammetry procedure is performed and a second image is made. The first and second images are compared to evaluate the ablating power of the laser. This procedure works well for imaging the artificial cornea before and after ablation, as initially described. The rastostereophotogrammetry is used on the reflective, pre-ablated substrate and the rastophotogrammetry is performed on the ablated substrate.

Therefore, rastophotogrammetry is useful in this type of surgical setting. Rastostereophotogrammetry may be performed on the cornea before deepithelialization or the rastophotogrammetry is performed on the deepithelialized cornea to determine a preablation corneal topography. Corneal ablation is performed and a repeat rastophotogram is made and compared to the first to validate the laser effect. One again, the purpose of this procedure is to attain the desired change in the refractive power of the cornea.

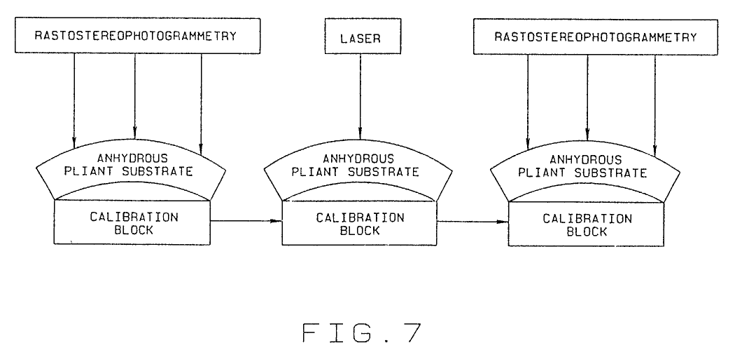

FIG. 7 illustrates the use of rastostereophotogrammetry, in addition to rastophotogrammetry, in the evaluation of a surgical laser using a novel, relatively anhydrous target substrate of the present invention. A novel target substrate is fastened to a holding block, as previously described. The target substrate is designed to be more efficiently ablated than a PMMA target substrate, lens implant or artificial cornea made from conventional materials, as previously described. Specifically, the target substrate is softer and ablates closer to the ablation rate of human corneal tissue. A simulated photorefractive keratectomy with an ultraviolet laser, for example as represented by the laser in the drawing, is used to very sensitively calibrate the laser. It generally is known that the human cornea typically ablates a rate that is dependent upon the laser's fluence. Further, the delivery system of the laser also effects the ablation rate for any given fluence. Specifically, a galvanometric delivery system that uses the purposeful, partial overlapping of adjacent pulses ablates human corneal tissue much more effectively than does a variable aperture (iris diaphragm) delivery system. Such a galvanometric delivery system is the subject matter of my co-pending application Ser. No. 08/616,288, filed Mar. 15, 1996. For example, I have determined that for a fluence level of 160 to 180 mJ/cm², the galvanometric delivery system ablates cornea about 4.3 times more effectively that PMMA. In contrast, at this fluence, the variable aperture delivery systems ablate cornea about 1.4 to about 1.7 times more effectively that PMMA. That is why it is essential that the target substrate of FIG. 7 have characteristics similar to those of human corneal tissue.

It is generally known that water adversely effects ablation efficiency by absorbing UV energy. Therefore, the target substrate must have controlled water content for calibration purposes. Thus, one target substrate of the present invention is an anhydrous, pliant substrate softer that PMMA. Such substrates include acrylic and silicone. Moreover, any soft, water free plastic is contemplated by the scope of the invention. Further, formed inorganic or organic gels will perform better than PMMA. An examples of such a formed organic gel is a cross-linked collagen and derivatives. Moreover, acrylic, collagen, agar and gelatin may be used.

The preferred substrates are of controlled water content and homogeneous. Because they ablate at a rate that is closer to corneal ablation (i.e. 0.24 to 0.50 microns/pulse at a UV laser fluence of 160 to 180 mJ/cm²), they are better suited than PMMA as a test substrate for laser calibration. In test ablations with a variable aperture system, acrylic ablated at 0.89 the rate of cornea. Thus, a −6 in cornea would be a −5.40 in acrylic. Also, a 6 mm ablation zone wherein 12 microns of central ablation depth represents one (1) diopter, a 2% error in acrylic represents 2% error of 72 microns, or 1.4 microns of central ablation depth, as opposed to as little as 0.96 micron in PMMA. Thus, the new target substrate represents approximately 50% increase in sensitivity as compared to PMMA. Using agar and gelatin mixtures of controlled water content, the ablation rate can be increased to more than 0.50 microns per pulse, thus making the target extremely sensitive for the purposes of calibration.

It will be appreciated that the procedure of FIG. 7 is analogous to the procedure of FIG. 6 except for the use of a preferred target substrate. Moreover, the target substrate of the present invention may be substituted for the calibration block, intraocular implant, artificial cornea or contact lens in any of the previously described procedures to give a more sensitive calibration and evaluation of the laser delivery service.

It will be appreciated by performing test ablations on the target substrate of the present invention will allow the surgeon to evaluate the effect the surgical laser will have on the human cornea. For example, the surgeon can perform a topographical analysis of the target substrate to determine the surface contour of the novel target substrate having ablative characteristics similar to those of the human cornea. Next, the surgeon can apply laser energy to the target substrate and perform a test ablation. Next, the surgeon can perform a second topographical analysis of the target substrate to determine the surface contour of the ablated target substrate. The surgeon then can compare the two surface substrates to evaluate the depth and area of ablation. In this manner the surgeon can evaluate the laser power, i.e. fluence produced by any given surgical laser and its effect on human cornea. The surgeon thus can modify his or her surgical technique in response to the evaluation.

I have determined that the improved calibration test target materials of the present invention, such as acrylic, collagen, agar, and gelatin which ablate at rates more equal (or in excess) to cornea than does PMMA, must be analyzed for topographic purposes with a non-contact profilemetry means. Such “soft” substrates can be distorted and the analysis yield erroneous results when contact techniques are used. Moreover, the contact techniques of surface profilemetry with a stylus-type apparatus are much slower than the non-contact techniques. In the clinical setting, the calibration process must me done in a timely manner.

It will be appreciated that the surgeon may use any of the illustrated or described methods of topographical analysis including, but not limited to rastophotogrammetry, rastostereography, placido-disc imaging, conventional lensometry and so forth, without departing from the scope of the invention.

Further, it will be appreciated that a number of other topographical analysis procedures may be used in place of the rastophotogrammetry of the illustrations. Other non-contact techniques of surface profilemetry can be substituted. For example, laser holography (Eye Technology, Minneapolis, Minn.), scanning laser ophthalmoscope (Laser Diagnostics, San Diego, Calif.), optical coherence tomography and scanning white light interferometry (PhaseShift, Inc., Phoenix, Ariz.) all can be used to evaluate and calibrate the ablation performance. Moreover, these same techniques can be used to evaluate and calibrate infrared lasers for refractive surgery, as well.

In another preferred embodiment, the evaluation and calibration data are used to improve the predictive value of a self-educating neural network, such as those commercially available (MindSight, Inc., Oshkosh, Wis.). Prior art use of neural networks has included preoperative data, such as patient sex, race and age, refractive error, and concurrent medication. The use of the topographical data obtained through the above-described methods significantly improves the predictive value of a neural network. Laser performance is an important variable affected by such things as laser head function, optic degradation and alignment, ambient humidity, and software (algorithm) accuracy. The present invention simulates a proposed photorefractive keratectomy (PRK) and then accurately measures the ablation profile by a non-contact method of topographical analysis, as described above. This data can be included in the preoperative information of a neural network to enhance the network's ability to suggest alterations in the proposed laser treatment to reach the desired refractive goal. For example, the ablation profile of the simulated PRK in the appropriate test target substrate may show that the laser is ablating 4% too much material. This information is included in the data input of the neural network along with other preoperative data, such as patient age, sex, race, hormonal status and corneal topography.

Various changes or modifications can be made in the foregoing description and accompanying drawings without departing from the scope of the appended claims. Therefore, the foregoing description and drawings are intended to be illustrative only and should not be viewed in a limiting sense.

1. A method of evaluating a surgical laser for use in ophthalmologic surgery comprising the steps of:
performing a first evaluation of the topography of a target substrate to determine the surface contour of the target substrate, said target substrate having laser ablating characteristics substantially similar to a human cornea;
applying a laser beam to said target substrate;
ablating said target substrate with said laser beam;
performing a second evaluation of the topography of the target substrate to determine a second surface contour of the target substrate;
comparing the first surface contour with the second surface contour; and
evaluating the effect of said laser beam on a human cornea based upon the comparison of the first and second surface contours.

2. The method of claim 1 wherein the first and second evaluations of the topography of the target substrate is performed by a non-contact technique for evaluating topography selected from the group comprising laser holography, scanning laser ophthalmoscope, scanning white light interferometry, and optical coherence tomography.

3. The method of claim 1 comprising the step of including the evaluation of the effect of said laser beam on human cornea in a self-educating neural network.

4. A non-contact method of performing surface profilemetry including rasterstereography, videokeratography, laser holography, scanning laser ophthalmoscope, optical coherence tomography, or scanning white light interferometry in a simulated laser ablation of a test substrate to evaluate an ultraviolet or infrared laser used in photorefractive keratectomy.

5. A method of enhancing the predictive value of a self-education neural network comprising the steps of:
performing a first non-contact evaluation of the topography of a target substrate to determine the surface contour of the target substrate, said target substrate having laser ablating characteristics substantially similar to a human cornea;
applying a laser beam to said target substrate;
ablating said target substrate with said laser beam;
performing a second non-contact evaluation of the topography of the target substrate to determine a second surface contour of the target substrate;
comparing the first surface contour with the second surface contour; and
evaluating the effect of said laser beam on a human cornea based upon the comparison of the first and second surface contours; and
including the evaluation of the effect of said laser beam on human cornea in a self-educating neural network.

6. The method of claim 5 wherein the non-contact evaluation of the topography is performed by a procedure selected from the group comprising rasterstereography, videokeratography, laser holography, scanning laser ophthalmoscope, optical coherence tomography, and scanning white light interferometry.

7. A method of evaluating a surgical laser for use in ophthalmological surgery comprising the steps of:
selecting a test substrate which ablates at the rate approximately equal to or in excess of the ablation rate of the cornea;
performing a first non-contact surface topographical analysis of the substrate;
ablating the substrate; and
performing a second non-contact surface topographical analysis of the substrate; and
comparing the second topographical analysis to the first topographical analysis to evaluate the surgical laser.

8. The method of claim 7 wherein the substrate is selected from the group containing acrylic, collagen, agar and gelatin.
